(19) United States
(12) Patent Application Publication
(10) Pub. No.: US 2023/0061426 A1
(43) Pub. Date: Mar. 2, 2023

(54) ANTIOXIDANT POLYMERIC BIOMATERIAL FOR TISSUE ENGINEERING AND METHODS OF USING SAME

(71) Applicant: Trilliant Surgical LLC, Houston, TX (US)

(72) Inventor: Tyler Joseph Touchet, Cypress, TX (US)

(21) Appl. No.: 17/736,256

(22) Filed: May 4, 2022

Related U.S. Application Data
(60) Provisional application No. 63/301,208, filed on Jan. 20, 2022.
(60) Provisional application No. 63/184,740, filed on May 5, 2021.

(51) Int. Cl. A61L 27/26; A61L 27/56; A61L 27/54; A61K 38/06; A61K 38/18

(52) U.S. Cl. CPC A61L 27/26; A61L 27/56; A61L 27/54; A61K 38/06; A61K 38/1841; A61L 2300/25; A61L 2430/02; A61L 2400/06; A61L 2430/24; A61L 2300/414

(57) ABSTRACT

Provided are thiol-ene polymer networks which can reduce the ROS species that contribute to delayed bone healing and fusion. Furthermore, patients that suffer from neuropathic comorbidities such as diabetes suffer from a diminished healing capacity. An increase in proinflammatory factors and the high presence of reactive oxygen species (ROS) present in diabetics are linked to lower fusion rates. To this end, there is a need for a clinically relevant bone graft to promote bone fusions in patients with neuropathic comorbidities. Incorporating thiol-ene networks for bone scaffolds has demonstrated increased osteogenic biomarkers over traditional polymeric materials and act as antioxidants. Thiol-ene networks offer improved bone grafts for diabetic patients by reducing the number of hydroxyl radicals associated with neuropathic comorbidities. These networks are particularly well suited in promoting healing in patients with Type II Diabetes or other conditions exacerbated by ROS-mediated damage.

### RELATED APPLICATION

This application is related to, and claims priority to U.S. Provisional Patent Application Ser. No. 63/184,740, filed on May 5, 2021, and entitled ANTIOXIDANT POLYMERIC BIOMATERIAL FOR TISSUE ENGINEERING AND METHODS OF USING SAME, and U.S. Provisional Patent Application Ser. No. 63/301,208, filed on Jan. 20, 2022, and entitled ANTIOXIDANT POLYMERIC BIOMATERIAL FOR TISSUE ENGINEERING AND METHODS OF USING SAME, the contents of which are hereby incorporated by reference in their entireties.

### FIELD OF THE INVENTION

This application relates to a material used to reduce abnormal oxidative stress in the body. Also disclosed are methods of using the material for therapeutic indications.

### BACKGROUND

Oxidative stress is a normal component of physiological function. However abnormal levels of reactive oxygen species can disrupt normal physiological pathways and cause disease states to manifest. Osteoclasts are multinuclear, hematopoietic cells of the monocyte and macrophage lineage. Osteoclasts demineralize bones through extracellular bone dissolution, a process involving the secretion of hydrolytic enzymes and protons and the generation of ROS. Oxidative stress, i.e. toxicity inflicted by ROS, can play a significant role in bone disease and has been implicated in such conditions as osteoporosis, periodontal disease, osteopenia, and osteolytic bone disease to name a few.

For individuals with metabolic disorders that promote osteoclastic behavior, for example, a decreased capacity to heal broken bones or recover from orthopedic surgeries has been observed. This leads to further patient complications such as infection, amputation and in serious cases death.

Type II Diabetes (T2D) affects up to 21 million people in the United States and continues to increase each year. Patients with T2D have a decreased ability to heal after a bone fracture or reconstructive surgery. Delayed healing or non-union can be attributed to several factors, such as reactive oxygen species (ROS) like hydrogen peroxide, that promote osteoclastic activity over osteoblastic.

The current standard of care for at-risk patients is to use more hardware to increase fixation to achieve boney fusion. Unfortunately, these methods have not demonstrated clinical success and in the case of extremities, failed fixation in T2D patients often leads to amputation.

The ideal bone graft can be characterized by the ability to promote bone fusion despite the native environment, degrade at a rate complementary to neotissue formation, and maintain mechanical integrity throughout the remodeling process. Specific to T2D, modulation of the biological response to decrease inflammation and oxidative stress could return tissue back to homeostasis. By fabricating a synthetic bone graft with the antioxidant moieties, the shortcomings of current solutions may be addressed.

There remains a need for treating oxidative stress and its impact on abnormal osteoclastic behavior.

It should be noted that this Background is not intended to be an aid in determining the scope of the claimed subject matter nor be viewed as limiting the claimed subject matter to implementations that solve any or all of the disadvantages or problems presented above. The discussion of any technology, documents, or references in this Background section should not be interpreted as an admission that the material described is prior art to any of the subject matter claimed herein.

### SUMMARY

It is understood that various configurations of the subject technology will become apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, drawings and detailed description are to be regarded as illustrative in nature and not as restrictive.

A method of performing arthrodesis in an individual in need thereof is described. The method may include providing an artificial bone graft. Advantageously, the bone graft includes an effective amount of a methacrylate monomer and an effective amount of a thiol-containing macromer. The method may also include implanting the artificial bone graft at the region where bone fusion is desired. The bone graft has antioxidant properties and is adapted to scavenge reactive oxygen species. The bone graft may also reduce osteoclastic activity at the fusion site.

The method of performing arthrodesis is well-suited for treating individuals for whom conventional arthrodesis have resulted in sub-optimal results and may have required salvage procedures. The individuals may be Type 2 diabetics. Alternatively or in addition, the individual may be osteoporotic. In some instances, the individual suffers from cancer. The arthrodesis may be an MPJ fusion procedure.

In one aspect, the methacrylate monomer is 1, 4-budanediol-dimethacrylate, diurethane dimethacrylate, or combinations thereof. In another aspect, the thiol-containing macromer is a multi-functional mercaptopropionate, mercaptoacetate, or combinations thereof.

The methacrylate monomer and thiol-containing macromer may be present at a ratio of 50:50, 60:40, or 70:30.

An oxidatively responsive polymeric scaffold for promoting bone fusion is likewise provided. The scaffold includes an effective amount of a methacrylate monomer; and an effective amount of a thiol-containing macromer. The polymeric scaffold includes sulfide linkages which are configured to sequester reactive oxygen species molecules. Advantageously, the scaffold has a degradation rate which is complementary to the rate of bone healing.

The methacrylate monomer may be 1,4-butanediol dimethacrylate or diurethane dimethacrylate. The thiol-containing macromer may be a multi-functional mercaptopropionate, mercaptoacetate, or combinations thereof. Optionally, the scaffold may also include bioactive agent coupled to a thiolated group. Suitable bioactive agents may include an Arginine-Glycine-Aspartate peptide sequence or a cytokine such as TGF-β.

In some aspects, the ratio of methacrylate monomer to thiol-containing macromer is 50:50. In other aspects, the ratio is 60:40. In still other aspects, the ratio is 70:30.

The scaffold may be formulated as a bone graft, an injectable gel, or as an artificial joint surface having a hydrophilic layer within a matrix.

In yet another aspect, the scaffold includes a plurality of pores having an average pore size between about 80 μm and 300 μm.

### BRIEF DESCRIPTION OF THE DRAWINGS

Various embodiments are discussed in detail in conjunction with the Figures described below. These embodiments are for illustrative purposes only and any scale that may be illustrated herein does not limit the scope of the technology disclosed. These drawings include the following figures, in which like numerals indicate like parts.

FIG. 1 is a chemical structure of a compound used to evaluate the atomic charge, wherein specific areas of interest include the outer beta-thioester group (encircled as ovals) and the pentaerythritol core indicated as encircled in the center of the compound.

FIG. 2 is a graphical representation of an Attenuated Total Reflectance Fourier Transform Infrared (ATR-FITR) spectroscopy of resin compositions.

FIG. 3 is a graphical illustration of the storage modulus of thiol-ene photopolymers.

FIG. 4 is a graphical illustration of the Tan δ of thiol-ene photopolymers.

FIG. 5 is a bar graph of cell viability of resin compositions per ISO 10993-5 compared to the acceptance criteria of 70%.

FIG. 6 is another bar graph illustrating the theoretical atomic charge (e) of the central carbonyl atoms from the compound as illustrated in FIG. 1.

FIG. 7 is a graph showing the percent mass loss of scaffold material under accelerated oxidative conditions.

FIG. 8 is a graph showing water uptake of scaffold material under accelerated oxidative conditions.

FIG. 9 is a graph showing the percent mass loss of scaffold material under accelerated hydrolytic conditions.

FIG. 10 is a graph showing water uptake of scaffold material under accelerated hydrolytic conditions.

FIG. 11 is a graph illustrating a set of ATR-FTIR waveforms of thiol-ene photopolymers subjected to accelerated oxidative degradation at 3 days and 18 days compared to the untreated control.

FIG. 11 is a graph illustrating another set of ATR-FTIR waveforms of thiol-ene photopolymers subjected to accelerated oxidative degradation at 3 days and 18 days compared to the untreated control.

FIG. 12 is a graph illustrating another set of ATR-FTIR waveforms of thiol-ene photopolymers subjected to accelerated oxidative degradation at 3 days and 18 days compared to the untreated control.

FIG. 13 is a graph illustrating another set of ATR-FTIR waveforms of thiol-ene photopolymers subjected to accelerated oxidative degradation at 3 days and 18 days compared to the untreated control.

FIG. 14 is a graph illustrating another set of ATR-FTIR waveforms of thiol-ene photopolymers subjected to accelerated oxidative degradation at 3 days and 18 days compared to the untreated control.

FIG. 15 is a graph illustrating another set of ATR-FTIR waveforms of thiol-ene photopolymers subjected to accelerated oxidative degradation at 3 days and 18 days compared to the untreated control.

FIG. 16 is a graph illustrating another set of ATR-FTIR waveforms of thiol-ene photopolymers subjected to accelerated oxidative degradation at 3 days and 18 days compared to the untreated control.

FIG. 17 is a graph illustrating a set of ATR-FTIR waveforms of thiol-ene photopolymers subjected to accelerated hydrolytic degradation at 42 days compared to untreated control.

FIG. 18 is a graph illustrating another set of ATR-FTIR waveforms of thiol-ene photopolymers subjected to accelerated hydrolytic degradation at 42 days compared to untreated control.

FIG. 19 is a graph illustrating another set of ATR-FTIR waveforms of thiol-ene photopolymers subjected to accelerated hydrolytic degradation at 42 days compared to untreated control.

FIG. 20 is a graph illustrating another set of ATR-FTIR waveforms of thiol-ene photopolymers subjected to accelerated hydrolytic degradation at 42 days compared to untreated control.

FIG. 21 is a graph illustrating another set of ATR-FTIR waveforms of thiol-ene photopolymers subjected to accelerated hydrolytic degradation at 42 days compared to untreated control.

FIG. 22 is a graph illustrating another set of ATR-FTIR waveforms of thiol-ene photopolymers subjected to accelerated hydrolytic degradation at 42 days compared to untreated control.

FIG. 23 is a bar graph illustrating antioxidant activity of thiol-ene photopolymers when submerged in hydroperoxide.

FIG. 24 is a SEM microscope photo of the surface of a biomaterial embodiment as described herein.

### DETAILED DESCRIPTION

The following description and examples illustrate some exemplary implementations, embodiments, and arrangements of the disclosed invention in detail. Those of skill in the art will recognize that there are numerous variations and modifications of this invention that are encompassed by its scope. Accordingly, the description of a certain example embodiment should not be deemed to limit the scope of the present invention.

Implementations of the technology described herein are directed generally to the reduction of an oxidative stress environment in diseased states. Exemplary disease states can include diabetes, osteoporosis, and cancer as tumors have been associated with high levels of ROS. The reduction of the oxidative stress environment allows for normal physiological function to occur such as bone fusion. Additionally, the reduction of an oxidative stress environment can mitigate the initial inflammatory response associated with implants.

Several types of ROS are essential to normal cellular function including superoxides, hydrogen peroxide, and hydroxyl radicals. Normal physiology closely regulates the amount of ROS production with antioxidants to maintain homeostasis. However, in metabolic diseases such as T2D, there is an imbalance in ROS, in particular, hydrogen peroxide. Extracellular concentration of hydrogen peroxide above 100 μM has been shown to have a negative impact on bone formation. Increases in proinflammatory factors and the high presence of ROS present in diabetics upregulate the expression of nuclear factor-κB (NF-κB) attributed to oxidative stress. An abundance of NF-κB is linked to an increase of osteoclastic differentiation and lower fusion rates.

The invention is based, in part, on the surprising discovery that by sequestering excess reactive oxygen species in polymeric biomaterial, homeostasis can be restored, and bone healing and fusion can occur. In some embodiments, the sequestration of excess reactive oxygen species in polymeric biomaterial promotes fusion of bone segments, particularly with respect to individuals suffering from metabolic disorders. Likewise, the invention can be applied to any application where reactive oxygen species need regulation such as wound healing, drug therapeutics, and artificial joints.

First metatarsasophalangeal joint (MPJ) arthrodesis is a common podiatric surgery involving the fusion of the first metatarsal and the first phalanx. In 2014, there were over 89,000 (HCUP) cases, with approximately 33% failure rate, which is exacerbated by comorbidities. A failed primary arthrodesis is followed up by a “salvage” procedure, typically involving the use of more extensive hardware and bone grafts to recreate the first metatarsal. Unfortunately, salvage surgeries have a similar rate of failure attributed to delayed healing, bone graft dissolution, and the lack of bone ingrowth. Allografts or synthetic grafts may be used to restore length but unsuccessful salvage can lead to anatomical deformity and in some instances, amputation. Current techniques fail to establish sustained compression to promote boney fusion. Additionally, such techniques further fail to address the cause of the non-union. Furthermore, patients suffering from neuropathic comorbidities such as diabetes suffer from a diminished healing capacity. An increase in proinflammatory factors and the high presence of reactive oxygen species (ROS) present in diabetics are linked to lower fusion rates. To this end, there is a need for a clinically relevant bone graft to promote bone fusions in patients with neuropathic comorbidities.

Reduced healing capacity is of particular interest when performing fracture repair or MPJ fusion procedures in extremities such as the foot. The current standard of care for at-risk patients is to use more hardware to increase fixation to aid boney fusion. Unfortunately, these methods have not demonstrated superior clinical success and in the case of extremities, failed fixation of T2D patients often leads to amputation. The use of bone grafting in T2D patients has become more popular in attempts to increase the likelihood of bone fusion. Current bone grafting techniques utilize either allografts, demineralized bone matrix, or ceramic-based pastes to serve as a filler and promote fusions. These bone graft options have demonstrated clinical success in the foot with open base wedge procedures. However, currently available grafts are not well suited for salvage surgery in patients with neuropathic comorbidities, and they achieve sub-optimal results.

Both allografts and synthetic grafts can be used for restoring the MPJ length. Allografts are available in a variety of geometries and often require cryopreservation. Synthetic grafts, often made from ceramics such as hydroxy apatite or Bioglass®, are moldable to fit the void but are not truly resorbable. However, current grafts do not address the underlying pathology, particularly with at risk patient groups such as diabetics and osteoporotic patients. Indeed, these patients are often contraindicated for treatment as they have a lower likelihood of success and limited healing capacity resulting in increase of pro-inflammatory factors. These pro-inflammatory factors can include the high presence of Reactive Oxygen Species (ROS) such as hydrogen peroxide and the upregulation of nuclear factor-kB (NF-kB), which results in osteoclastic behavior. Low levels of graft incorporation may be attributed to the failure of the grafts to influence pathophysiology. To this end, there is a need for a clinically relevant bone graft to promote bone fusions in patients with T2D which is addressed by the disclosed scaffold. In some embodiments, the thiol-ene scaffold disclosed herein can be used as bone graft that focuses on achieving fusions in metabolically diseased patients.

Low levels of graft incorporation may be attributed to the failure of the grafts to influence pathophysiology. To this end, there is a need for a clinically relevant bone graft to promote bone fusions in patients with T2D.

In one aspect, improved biomaterial for promoting bone fusion is provided. The antioxidant polymeric biomaterials or scaffolds described herein are biodegradable, porous, mechanically strong, configured to be sterilized without compromising function, augment current surgical techniques, and possess an antioxidant effect.

The ideal bone graft for a salvage surgery can be characterized by the ability to promote bone fusion despite the native environment, degrade at a rate complementary to neo tissue formation, and maintain mechanical integrity throughout the remodeling process.

Modulation of the biological response to decrease inflammation and oxidative stress return tissue back to homeostasis. Additionally, graft degradation rates that are complementary to the healing rate are also highly desired. If the graft degrades at a rate faster than new bone is generated, fusion will not occur. Conversely, if the rate of degradation is slower than the healing rate, delayed fusion and morphological irregularities manifest. Maintaining the mechanical integrity of the graft through the regenerative process prevents failures, such as nonunion or irregular morphologies.

Provided herein are polymeric scaffolds based on thiol “click” chemistry. Thiol “click” chemistry advantageously reacts with a variety of functional groups under mild conditions. Thiolated groups allow for the facile coupling of bioactive agents, such as the peptide sequence RGD (Arginine-Glycine-Aspartate) or cytokines such as Transforming Growth Factor β (TGF-β). Additionally, thiol-methacrylate nanocomposite systems for use in biomedical applications exhibit excellent colloidal stability up to 11 months in air but not in hydrolytic or oxidative environments. The incorporation of thiol-ene networks for bone scaffolds increases alkaline phosphatase and osteocalcin expression over poly(ε-caprolactone) hydroxyapatite scaffolds (PCL: HA) scaffolds. Furthermore, thiol-ene networks can act as antioxidants. Sulfide linkages within the network have an ability to consume radical oxygen to create sulfoxide and sulfone groups. These unique properties of thiol-ene networks make them particularly well suited as bone grafts in patients with high levels of oxidative stress. Photopolymers that are oxidatively responsive via sulfide linkages can sequester ROS molecules at pathophysiologic levels spontaneously and trigger a predicable degradation mechanism. Thiol-ene thermoset polymers are disclosed herein which are particularly well-suited for use as bone grafts.

The use of chemical moieties to target excessive amounts of reactive oxygen species to decrease osteoclastic behavior and promote osteoblastic behavior are provided. As set forth in the Example section, chemical and oxidative degradation properties of a class of thiol-ene based thermoset polymers for use as bone grafts are described. Additionally, the thiol-ene polymer networks were evaluated in terms of their oxidative responsiveness and ability to scavenge hydrogen peroxide at pathophysiologic levels.

Incorporating thiol-ene networks for bone scaffolds has demonstrated increased osteogenic biomarkers over traditional polymeric materials. Furthermore, thiol-ene networks can act as antioxidants. Sulfide linkages within the network have an ability to consume radical oxygen to create sulfoxide and sulfone groups. These unique properties of thiol-ene networks make them a promising candidate as bone grafts for diabetic patients. A thiol-ene biomaterial is provided to address the current limitations of MPJ fusion in diabetics. Notably, thiol-ene based materials described herein are shown to reduce the number of hydroxyl radicals associated with neuropathic comorbidities.

As will be described in greater detail below, the scaffold disclosed herein possesses characteristics which promote therapeutic success. Such characteristics include, without limitation, the ability to be biodegradable. In certain embodiments, the scaffold can fully degrade within 12-24 months. Other characteristics include non-toxicity and ability to be sterilized without compromising function. The antioxidant properties of the scaffold disclosed herein have biomedical applications. The antioxidants reduce the amount of ROS in the body by sequestering radical species. Incorporation of antioxidant groups into polymers creates a class of stimuli responsive materials which can be useful in the development of drug delivery based on a ROS environment, tissue-engineered scaffolds, and antiadhesion barriers. Advantageously, the scaffold is mechanically strong enough to be handled by a surgeon, can be sterilized without compromising function, and can augment current surgical techniques. Moreover, the thiol-ene scaffold has a sustained antioxidant effect of greater than six months and up to about one year. The antioxidant effect includes an ability to sequester approximately 1 mmol of hydrogen peroxide.

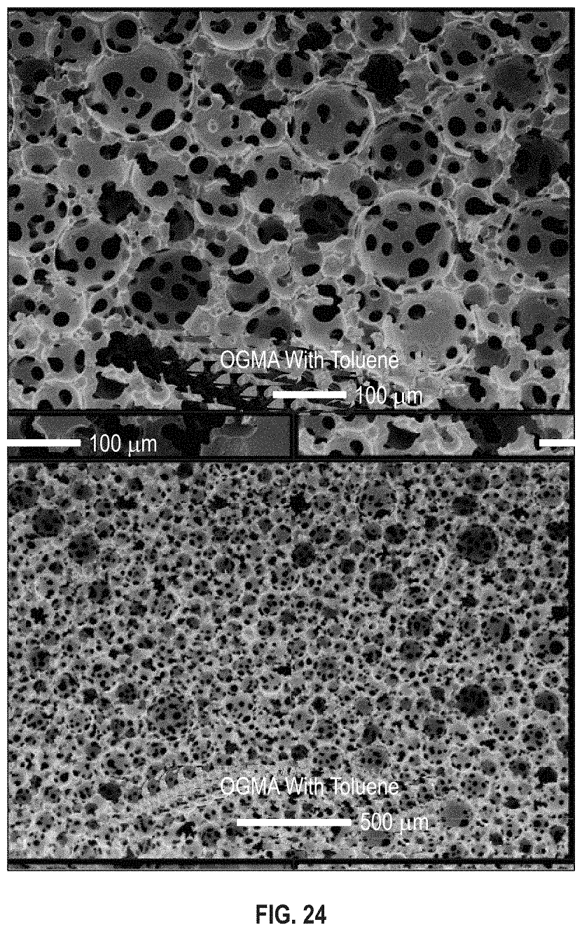

As best illustrated in FIG. 24, the polyHIPE fabrications produce highly stable scaffolds that are also highly porous. FIG. 24 is a SEM image of the scaffold surface at different magnifications and illustrates the plurality of interconnected voids and porosity of the scaffold. In certain embodiments, the scaffolds have a porosity of over 60%. In other embodiments, the porosity is above 70%. Porosity characteristics include interconnected voids of approximately 50 μm and between about 100-1000 μm substantially heterogenous sized pores. In some embodiments, the void size average is between 30 to 60 μm and the average pore size is between around 80 μm and 300 μm. The pore profile of the scaffold possesses a modulus of between about 1000 kPa and 3000 kPa and a compressive strength (yield or 10% strain) of between about 120 and 250 kPa. The pore size is indirectly proportional to the modulus of scaffold.

Several embodiments are described with the primary focus of a bone graft. It should be noted, however, that applications are not limited to bone grafting. Indeed, application can be extended to an injectable gel, a film or coating, artificial joint surfaces, or adhesion barriers. Embodiments have a foundation in inherent antioxidant behavior as a part of the polymer backbone and not through additives or pendent groups. Changes in properties can be achieved through knowledge of structure properties relationships. For example, to go from a bone graft to an injectable gel, the polymer may change in MW between crosslinks, the type of cross-links (physical v. chemical), and in hydrophilicity. These variables can be modulated without an impact to the antioxidant functional groups.

A tissue-engineered scaffold based on thiol-ene chemistry is provided. The thiol-ene scaffold is particularly well-suited for addressing the need for bone-graft materials, particularly for use in salvage Metatarsophalangeal joint (MPJ) procedures. Salvage procedures of the first metatarsophalangeal joint (MPJ) often require bone grafts to restore normal anatomy and function. A unique microenvironment characterized by high levels of reactive oxygen species (ROS) and a low pH results in a delay of bone formation and favors osteoclast activity. Current surgical techniques leverage traditional bone grafting materials such as allografts or demineralized bone matrix (DBM). Unfortunately, current options have a high rate of dissolution and lack the mechanical properties needed before ossification occurs.

To evaluate the structure-property relationships of the thiol-ene networks, a library of compositions were fabricated. Network components comprise monomers with biomedical applications such as bone scaffolds, shape memory polymers, hydrogels, and photoinitiators as set forth in the Table 1. Compositions were evaluated and assessed by varying crosslink density, thiol content and number of ester linkages.

TABLE 1
Monomer selection for thiol-ene based scaffolds and composition of current
off the shelf polymers used for bone tissue engineering

| Monomer Name | |
|---|---|
| & Abbreviation | Chemical Structure |
| 1,4-Butanediol Dimethacrylate (BDMA) | 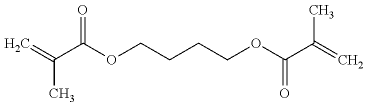 [SMILES: C=C(C)C(=O)OCCCCOC(=O)C(=C)C] |
| Pentaerythritol- Tetrakis(3- mercptopro- pionate) (PTMP) | 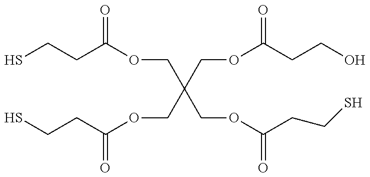 [SMILES: O=C(CCO)OCC(COC(=O)CCS)(COC(=O)CCS)COC(=O)CCS] |
| Diurethane Dimethacrylate (DUDMA) | 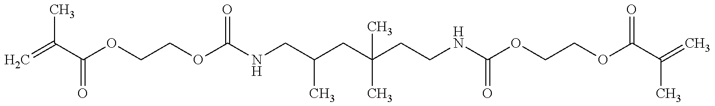 [SMILES: C=C(C)C(=O)OCCOC(=O)NCCC(C)(C)CC(C)CNC(=O)OCCOC(=O)C(=C)C] |
| Poly(ϵ- caprolactone) tetra (3-mercpto- propionate) (PCL4MP) | 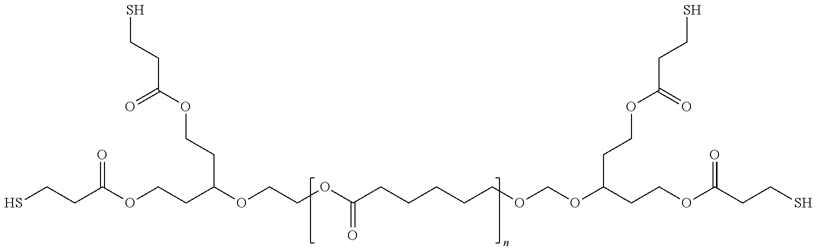 |
| Glycol Dimercapto- acetate (GMA) | 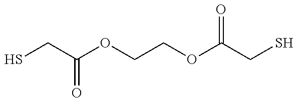 [SMILES: O=C(CS)OCCOC(=O)CS] |
| Phenylbis(2,4,6- trimethyl- benzoyl) phosphine oxide (BAPO) | 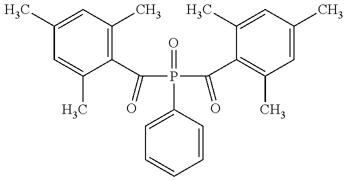 |
| Azobisiso- butyronitrile (AIBN) | 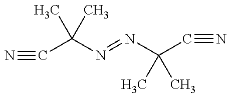 |

Disclosed herein is a solution to overcome the shortcomings of current materials via bone tissue engineering. Bone tissue engineering strategies focused on polymeric scaffolds show promise in providing an alternative solution for traditional bone grafting materials. However, traditional polymer scaffolds based on polyesters have limited tunability. Alternatively, scaffolds based on thiol-ene “click” chemistry demonstrate the ability to tailor polymer properties, such as degradation and as-implanted mechanical properties, for targeted applications. As a result, thiol-ene based scaffolds for bone tissue engineering offer promise for salvage MPJ procedures.

Thiol-Ene Polymers as Scaffold Materials

To determine the optimal material composition, the impact of varying the crosslink density, thiol content, and the number of ester linkages is assessed. The chemical properties of the thiol-ene networks can be characterized by analytical methods such as Attenuated Total Reflectance Fourier Transform Infrared Spectroscopy (ATR-FTIR), Differential Scanning calorimetry (DSC) and Dynamic Mechanical Thermal Analysis (DMTA). Mechanical properties can be analyzed by using DMTA and static compression testing. Additionally, in vitro accelerated degradation testing and cytotoxicity studies demonstrate clinical relevance.

A method of fabricating Thiol-ene Photopolymers (TPP), bone void fillers, can be achieved through mixing the compositions in the weight percentages set forth in Table 2 below. The method is described as follows: Emulsion templated scaffolds are fabricated by using an overhead stirrer with a non-reactive polymer paddle. The monomers are added to a vessel and the paddle is set to between about 75-500 rpm. In some embodiments, the paddle rate is between about 100-300 rpm. In another embodiment, the paddle rate is about 300 rpm. Water is then added dropwise until the water phase exceeds 74% of the volume. The water percentage can range between about 74-99% and is preferably between about 80-90%. At this point, the monomer phase and water phase are in a state known as a high internal phase emulsion (HIPE). Once the water phase is completely incorporated, the HIPE can be cured in two stages, although one phase is acceptable. When using BAPO, the first cure is a 40 W UV cure at between about 365 nm-405 nm followed by a dual UV/heat cure for 30 min at 60° C. The cure time can range from about 5 minutes to 24 hours. After curing, the HIPE are thoroughly washed with isopropyl alcohol for approximately 1 hour in an ultrasonic bath. The cleaned scaffolds are then transferred into a vacuum oven at 50° C. for about 24 hours prior to use, further processing, or characterization.

One feature of the biomaterial described herein is a nucleophilic element, preferably Group 16 of the periodic table, incorporated into the polymer backbone (i.e., thiol [R—SH] that reacts to become a sulfide [R—S—R]). Upon exposure to hydroxyl radicals, consumption of the radical is achieved by the polymer backbone, wherein the sulfide linkage sequesters peroxides (i.e., sulfide is oxidized to a sulfoxide and subsequently a sulfone). This is also true of selenium or tellurium. In another embodiment, sulfur can be replaced by phosphorus. The change in nucleophilic element creates a more hydrophilic polymer allowing for water to penetrate the matrix and hydrolytically cleave ester moieties. Alternatively, as will be appreciated by a person of skill in the art, any hydrolytically labile group can be used.

As the surface of the polymer matrix degrades away, a new layer of the polymer is exposed that will be able to undergo the previously stated oxidation route until the polymer is completely dissolved. Advantageously, this happens are a rate complimentary to new tissue formation. This can be correlated to the amount of reactive oxygen species.

Material described herein can sequester hydrogen peroxide, the main, radical oxygen species, at levels relevant to the targeted pathologies and can promote better tissue healing (soft and hard) as compared to the current gold standard of treatment.

In another aspect of the invention, multifunction monomers such as primary thiols, mercaptans, acrylates, urethanes, allyl, vinyl, and methacrylates are employed as antioxidant polymeric biomaterial. Custom acrylated esters can be used or different polymerization techniques (i.e. chain-growth polymerization versus step-growth polymerization). Secondary thiols can also be used.

The biomaterial is particularly well suited for bone grafting application in the extremities. It will be appreciated that it can also be used in bone grafting applications in spine, long bone, and/or cranial facial regions. These thiol-based resins demonstrate mechanical properties suitable for bone grafting applications but may be suitable for other orthopedic indications where ROS-mediated damage otherwise impedes healing. The incorporation of sulfide groups creates a ROS-responsive network, which promotes bone healing.

As will be appreciated by a person of skill in the art, the biomaterials described herein can be formulated in a number of different ways:

Injectable Gel—This embodiment is an antioxidant gel that is intended to influence the interstitial environment or in wound healing applications. The embodiment can be used to restore homeostasis where oxidative stress levels need continual control. For example, the gel can be used in a diabetic ulcer to promote healing and the reduction of cellular death. Another example is to inject the gel into joint spaces, much like Vitamin D treatments, prior to any surgical intervention to create an ideal environment for healing.

A solid or semisolid injectable microgel scaffold comprising antioxidant polymeric biomaterial for biomedical applications such as wound healing is contemplated. In some embodiments, the antioxidant polymeric biomaterial is a thiol-ene photopolymer. Fabrication of injectable, therapeutic polymer gel scaffolds are well-characterized (See, eg. U.S. Pat. No. 8,277,832).

The injectable scaffold may be used for various applications, including a variety of medical applications involving inflammation or where ROS-mediated damage is present such as medical trauma treatment, post-surgical closure, burn injuries, inflammatory and hereditary and autoimmune blistering disorders, for example. In one or more embodiments, the scaffold is used as a tissue sealant (e.g., an acute wound-healing substance, surgical sealant, topical agent for partial thickness, full thickness, or tunneling wounds, pressure ulcers, venous ulcers, diabetic ulcers, chronic vascular ulcers, donor skin graft sites, post-Moh's surgery, post-laser surgery, podiatric wounds, wound dehiscence, abrasions, lacerations, second or third degree burns, radiation injury, skin tears, and draining wounds, and the like). In certain embodiments, the injectable gel scaffold is used to enhance the healing of skin wounds (e.g., surgical sites, burn wounds, ulcers).

Artificial Joint Surface—This embodiment is a biostable formulation (removal of ester moieties) that sequesters the reactive oxygen species from the original host response to create a thin hydrophilic layer within the polymer matrix to allow for a tenacious self-lubricating surface. Preferably, the biostable formulation of an antioxidant polymer biomaterial comprises thiol-ene based polymeric multiparticles. The formulation can be employed for tissue engineering matrices, wound dressings, bone repair or regeneration materials, or as a thin coating to an artificial joint.

Adhesion Barrier—This embodiment can be used in surgical settings where adhesions are likely to occur to provide a barrier between the two tissue types. The reduction of the reactive oxygen species acts to reduce the inflammation at the site. However, the primary mechanism of this embodiment is the change in hydrophilicity to allow for rapid degradation before tissue ingrowth.

Additional embodiments can include different methods of polymerization to include chain growth, step growth, RAFT, living polymerization, Thiol-ene “Click” chemistry, or chain transfer polymerization. Furthermore, the scaffolding can be in the form a polymer monolith, electrospun fibers, wet-spun fibers, melt-spun fibers, gas foamed scaffold, freeze-dried scaffold, salt-leached scaffold, emulsion templated scaffold, hydrogel, compression molded scaffold, reaction injected molded scaffold, and 3D-printed scaffold. It should be noted that the use of scaffold may refer to a monolith that is non-porous or porous. Advantageously, the invention is porous with homogenous pores ranging from 100 to 500 microns.

Features of the invention are illustrated, in nonlimiting fashion, by the following examples. These examples are illustrative only and are not intended to limit the scope of the claims. Methods described below can be optimized using empirical techniques well known to those of ordinary skill in the art. Moreover, artisans of ordinary skill in the art would be able to use the teachings described in the following examples to practice the full scope of the claims:

### EXAMPLES

We evaluated photopolymers that are oxidatively responsive via sulfide linkages and their ability to sequester ROS molecules at pathophysiologic levels spontaneously and trigger a predicable degradation mechanism. In this study, we report the preliminary findings on the chemical, mechanical, and degradation properties of a class of thiol-ene based thermoset polymers for use as bone grafts. Additionally, the thiol-ene polymer networks were assessed in terms of their oxidative responsiveness and ability to scavenge hydrogen peroxide at pathophysiologic levels.

Materials & Methods

Chemicals and Reagents. All reagents were used as-is from commercial sources unless otherwise stated. Butanediol dimethacrylate (BDMA), diurethane dimethacrylate (DUDMA), pentaerythritol tetrakis3-mercaptopropionate (PTMP), polycaprolactone tetra(3-mercaptopropionate) (PCL4MP), glycol dimercaptoacetate, (GMA), phenylbis(2,4,6-trimethylbenzoyl) phosphine oxide (BAPO), high glucose Dulbecco's Modified Eagle's Medium (DMEM), trypsin-ethylenediaminetetraacetic (EDTA) solution, resazurin based In-Vitro toxicology assay kit, and newborn calf serum (NCS) were purchased from Sigma-Aldrich. Penicillin-Streptomycin solution (P/S) and Amphotericin B (Fungizone) were purchased from Cytiva. Dulbecco's phosphate buffered saline (DPBS) was purchased from Lonza. Cell culture media was made by adding 5.6 mL of P/S, 0.6 mL of fungizone, and 56 mL of NCS to 500 mL of DMEM. (PCL4MP) was provided by Bruno Bock Chemische Fabrik GmbH & Co (Germany)

Fabrication of Thiol-ene Photopolymers (TPP). Polymer disks approximately 12.0 mm in diameter and 2.5 mm thick or thin films approximately 10.0 mm×5.0 mm×2.5 mm (I×w×h) were fabricated by mixing thiol-containing macromers in various concentrations (Table 2). The thiol-containing macromer can be a multi-functional mercaptopropionate, mercaptoacetate, or combinations thereof,

TABLE 2
Formulation names and macromer compositions
for thiol-ene photopolymers fabricated.

| Formulation | Weight Percent (wt %) | | | | |
|---|---|---|---|---|---|
| Name | BDMA | PTMP | PCL4MP | DUDMA | GMA |
| TPP1a | 43.3 | 21.6 | 13.0 | 21.6 | — |
| TPP1b | 33.3 | 16.6 | 33.3 | 16.6 | — |
| TPP1c | 24.4 | 12.2 | 51.1 | 12.2 | — |
| TPP2 | 66.3 | — | — | 33.2 | — |
| TPP3 | 45.2 | 22.6 | — | 22.6 | 9.0 |
| TPP4 | 58.5 | — | 29.3 | — | 11.7 |

Methacrylate monomers included BDMA (226.27 g/mol) and DUDMA (470.56 g/mol). PTMP (488.66 g/mol), PCL4MP (1350.00 g/mol), GMA (210.27 g/mol) were used as the thiol-containing macromers. The methacrylate and thiol ratios were varied from 50:50 to 70:30. A control composition (TPP2) without thiol groups was fabricated in the same manner. Resin compositions were made by mixing macromers with 0.5 wt % photoinitiator (BAPO) until the resins were thoroughly mixed. Polymerization was achieved by exposing UV light at 425 nm for 5 minutes. The disks were then washed with isopropyl alcohol and dried under vacuum (32 mmHg) at 50° C. until a constant weight was achieved.

Accelerated Hydrolytic Degradation. Accelerated hydrolysis of the polymers was simulated by using an alkaline solution. 0.1 M NaOH solutions were made by adding 3.99 g of NaOH to 1000 mL of RO water, mixed well, and stored at 25° C. The polymer discs were submerged in 25 mL of solution in a sealed vial. The vials were placed in a 37° C. incubator (±1.0° C., measured continually with a thermocouple). Samples were incubated for 7 days before replenishing the solution and were weighed every week. At each time point, samples were removed, rinsed thoroughly in DI water, blotted with a Kim Wipe®, and dried in a 50° C. oven. Vacuum was pulled for 24 hours at 30 mm Hg. After removal from the oven, samples were weighed and returned to the vials with freshly replenished solution. Sample analysis is detailed below. The mass was converted to the remaining mass percentage, and the average change and standard deviation were recorded and plotted.

Accelerated Oxidative Degradation. The 20% H₂O₂ solution was used for the accelerated oxidative degradation solution. Films were submerged in 25 mL of solution in a sealed vial. The vials were placed in a 37° C. oven (±0.5° C., measured continually with a thermocouple). Samples were incubated for 7 days before replenishing the solution, checked daily to monitor solution levels, and were weighed every week. At each time point, samples were removed, rinsed thoroughly in RO water, blotted with a Kim Wipe®, weighed for water uptake, and dried in a 50° C. oven. Vacuum was pulled for 24 hours at 30 mm Hg. After removal from the oven, samples were weighed and returned to the vials with freshly replenished solution. Sample analysis is detailed below. Water uptake was calculated as a percentage from the original mass. The average and standard deviation were recorded and plotted. The mass was converted to the remaining mass percentage from the original mass, and the average change and standard deviation were recorded and plotted.

Atomic Charge Chemical Modeling. The atomic charge was calculated by creating model compounds containing a pentaerythritol core, a mercaptopropionate, and a reacted α-β saturated alkene at different oxidative states, FIG. 1. FIG. 1 illustrates the chemical structure of the model compound used to evaluate the atomic charge. The specific areas of interest include the outer β-thioester group (outer oval circled portions) and the pentaerythritol core (center circle). The atomic charge of the central carbonyl atom was calculated using methods described by Ionescu et al (Ionescu, C.-M.; Sehnal, D.; Falginella, F. L.; Pant, P.; Pravda, L.; Bouchal, T.; Vařeková, R. S.; Geidl, S.; Koča, J., AtomicChargeCalculator: interactive web-based calculation of atomic charges in large biomolecular complexes and drug-like molecules. Journal of cheminformatics 2015, 7 (1), 1-13, the contents of which are incorporated by reference in their entirety). The resulting values represent a quantitative expression of electron density. The values were recorded and plotted as a function of sulfur oxidation.

Chemical Characterization. Attenuated Total Reflectance Fourier Transform Infrared (ATR-FTIR) spectroscopy was performed using an infrared spectrometer (Bruker Alpha, Bruker). Spectral data was collected in absorption mode with a resolution of 1 cm⁻¹. OPUS software was used to examine spectra, identify peaks, and perform baseline and atmospheric corrections. Examinations were performed in triplicate to confirm the results. The location of the relevant peaks (carbonyl peak corresponding to the ester ranging from 1730 to 1735 cm⁻¹, the methacrylates (C═C) at ca 810/815 cm⁻¹ and 1632 cm⁻¹, and peaks associated with oxidation of the sulfide bond to sulfoxide (S═O) at ca 1036 cm⁻¹ and sulfones (S═O) ca 1128, 1311 cm⁻¹) were used to characterize the extent of degradation and the oxidative response. Quantitative conversion (DC %) was determined on representative samples using the changes to the peak at 815 cm⁻¹ normalized to 835 cm⁻¹ of uncured formulations and fully processed resins.

Thermal Mechanical Characterization. Dynamic Mechanical Thermal Analysis (DMTA) was conducted with a Q800 DMA (TA Instruments) on thin-film samples of approximately 5 mm in width and a length of 10 mm. Specimens were subject to a temperature ramp from −20° C. to 120° C. at a rate of 5° C./min while under a 0.1% cyclic strain at 1 Hz frequency. The glass transition temperature (T_{g}) was determined by taking the maximum value of the tan delta peak. The molecular weight between crosslinks was estimated using the following equation described by Flory and Rehner:

M_{c}=3RTρ/E′ (1)

M_{c} is the molecular weight between crosslinks, R is the universal gas constant, T is the temperature in Kelvin, rho (p) is the polymer density and E′ is the rubbery modulus.

Hydrogen Peroxide Consumption. A 200 μM H₂O₂ solution was used to simulate pathologic levels of hydrogen peroxide. Polymer disks were submerged in 25 mL of solution in a sealed vial. The vials were placed in a 37° C. oven (±0.5° C., measured continually with a thermocouple). Hydrogen peroxide levels were evaluated using a colorimetric Hydrogen Peroxide Assay Kit (CHEMetrics) upon initial insertion (t=0) and after 72 hours (t=72). Blank vials filled with the solution was used as a control. The concentrations were recorded, and the average value with the standard deviation of all five specimens was plotted.

Cytocompatibility. Cytotoxicity per ISO 10993-5 was completed for resin compositions where the bulk mechanical properties were considered comparable to trabecular bone. For each extraction, three sample disks (each with a surface area of 3.5 cm²) were added to 3 mL of cell culture media and allowed to incubate at 37° C. for 48 hours. While samples were undergoing extraction incubation, cells were seeded using 200 μL of fresh cell culture media in a 96 well plate at a density of 7500 cells/well. These cells were allowed to incubate for 24 hours to generate a subconfluent monolayer of cells. The cell culture media was then removed from each well and replaced with 200 μL positive control cell culture media, negative control cell culture media, or samples extraction cell culture media. Fresh cell culture media was used for positive controls, while cell culture media with 5% DMSO was used for negative control media. Six replicates were used for each control and test sample. Once control and test media had been added to the cells, they were allowed to incubate for 48 hours at 37° C. Following the incubation period, qualitative morphological scores were assigned to each well according to ISO-10993-5, and quantitative cell viability was assessed using a resazurin assay. For this assay, cell culture media was removed from each well and replaced with fresh media mixed 10% v/v with a resazurin assay solution. This mixed media was allowed to incubate at 37° C. for 3 hours, after which a plate reader was used to determine the fluorescence intensity with an excitation wavelength of 560 nm and emission wavelength of 590 nm. Cell viability for each sample was determined by using the positive control fluorescence as a 100% viability standard and a well with mixed media but no cells as 0% cell viability.

Results and Discussion

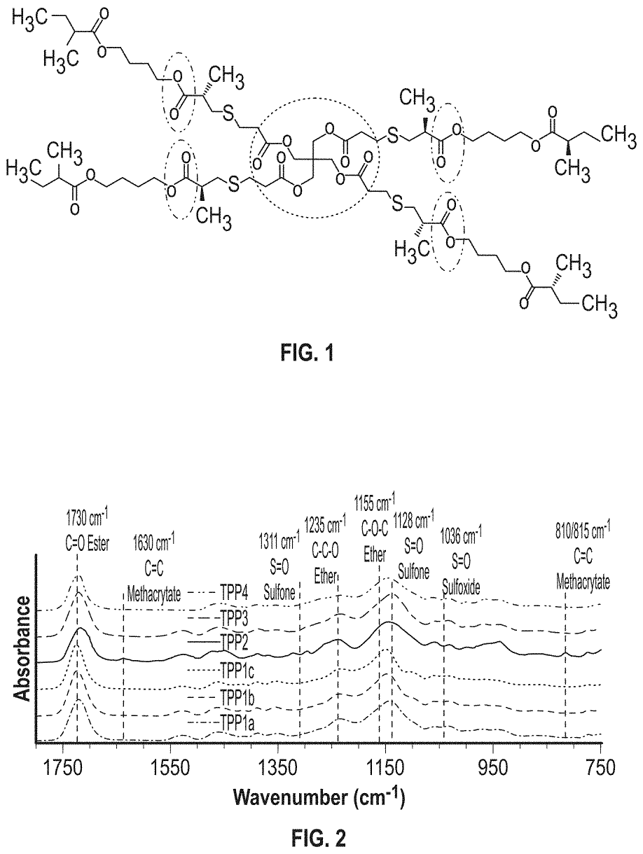

FTIR Characterization: All formulations produced optically clear disks approximately 12.5 mm in diameter and 2.5 mm in thickness. Fabrication of cured resin formulations showed a reduction in the free thiol and the unsaturated bond of methacrylate, indicating successful incorporation of thiols into the network, as bests observed in FIG. 2. FIG. 2 is an ATR-FTIR of all resin compositions with peaks of interest, methacrylate, and thiol peaks identified. The degree of conversion of processed resins demonstrated an increase over the methacrylate-only composition in TPP2, as set forth in Table 3 below. The results show the incorporation of thiol groups into methacrylate networks increases the conversion of the methacrylates. Higher conversion in the polymer reduces the number of unreacted monomers that can leach out of the polymer matrix during extraction. Post-cure processing, such as cleaning, can also influence toxicity by removing unreacted monomers, additives such as surfactants, and catalysts. Therefore, the cleaning method chosen may be relevant to the viability of the device once implanted. Ultrasonic washing using IPA may be an effective solvent for removing unreactive methacrylates and is applicable to the polymer networks described.

TABLE 3
Degree of conversion of thiol-ene photopolymers
after ultrasonic cleaning.

| Formulation | TPP1a | TPP1b | TPP1c | TPP2 | TPP3 | TPP4 |
|---|---|---|---|---|---|---|
| DC % | 98% | 89% | 97% | 70% | 99% | 99% |

Conversion, as expected, was lower in TPP2 at 70% whereas compositions containing thiols were greater, ranging from 89-99%. The unreacted methacrylate groups present, specifically in TPP2, are likely attributed to imperfections in the network leading to unreacted functional groups. Resin formulations that contained urethane linkages demonstrated broad carbonyl peaks around ˜1730 cm⁻¹ which can be attributed to hydrogen bonding of the urethane groups and ester linkages. Conversely, the urethane-free resin (TPP4) has a sharp carbonyl peak. Although traditionally a weak peak, free thiols were not detected based on the absence of the peak at 2500 cm⁻¹.

Thermal and Mechanical Characterization: The thermomechanical properties of the resin formulations were characterized through the analysis of storage modulus (E′) and tan δ curves. Both curves provide insight into the mechanical properties and morphology of the polymer networks as a function of temperature. Compositions that are mainly comprised of small monomers (TPP1a, TPP2, and TPP3) display a storage modulus in the gigapascal range at room temperature. However, the storage moduli shift into the megapascal range at body temperature, Table 4.

TABLE 4
Storage modulus and glass transition
values of thiol-ene photopolymers

| Formulation | E′ at 25° C. | E′ at 37° C. | T_{g} |
|---|---|---|---|
| Name | (MPa) | (MPa) | (° C.) |
| TPP1a | 1420 | 484 | 47 |
| TPP1b | 50 | 25 | 11 |
| TPP1c | 7 | 7 | −19 |
| TPP2 | 3015 | 2677 | 51 |
| TPP3 | 1318 | 175 | 38 |
| TPP4 | 349 | 166 | 27 |

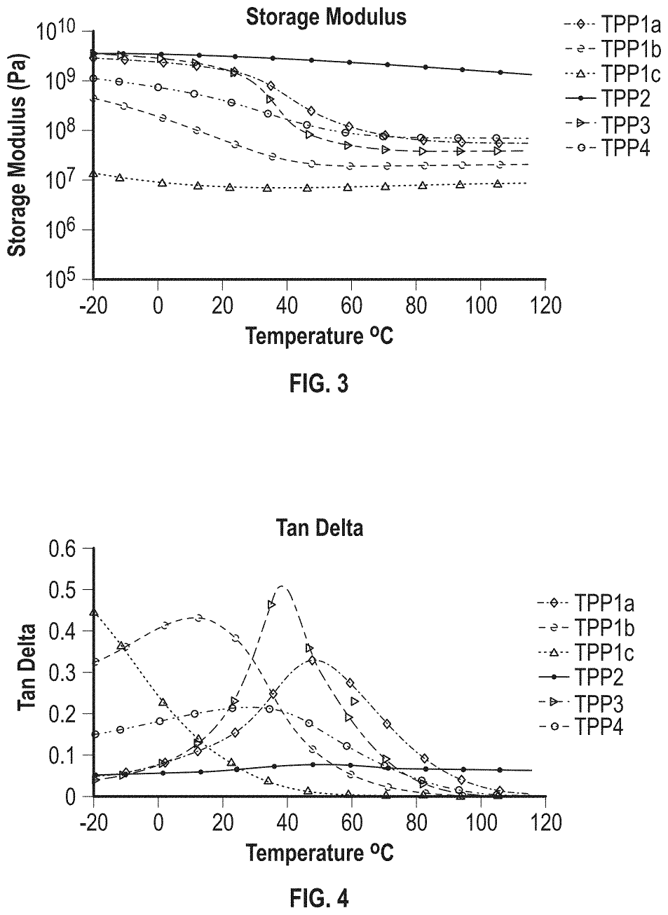

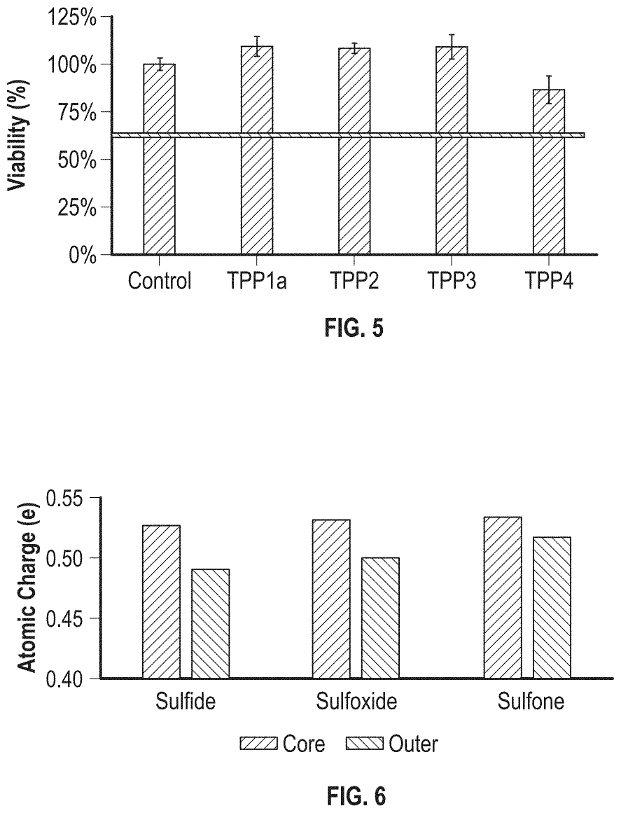

Despite the decrease in storage modulus values, TPP1a and TPP3 are relevant to trabecular bone with 484 MPa and 175 MPa, respectively. While outside of the gigapascal range at body temperature, the structural integrity of the bone graft is not anticipated to be compromised. The clinical application of the bone graft utilizes hardware such as a locking plate and compression screws to share the biomechanical load. The high storage modulus values are likely attributed to the ideal network formation and the influence of hydrogen bonding from the urethane groups. The glass transition onsets were characterized by a decrease in the storage moduli or an increase in tan δ, as best illustrated in FIGS. 3 and 4, which plot the Storage Modulus (FIG. 3) and Tan-δ (FIG. 4) of thiol-ene photopolymers.

As expected, changes in chain mobility and crosslink density led to changes in the glass transition temperature. The increases in chain mobility and decreases in crosslink density are demonstrated in the response of TPP1a, TPP1b, and TPP1c. For these formulations, the amount of PCL4MP increases from 13.0 wt % to 51.1 wt %. As the amount of PCL4MP increases, the Tg shifts to lower temperatures and is less pronounced. This is expected as the increase in PCL leads to a network that takes on the characteristics of PCL. This phenomenon is further confirmed by a decrease in the rubber modulus, which is related to the molecular weight between crosslinks of the polymer network or the crosslink density. The molecular weight between crosslinks from TPP1a and TPP1c increases over seven-fold from ˜177 g/mol to ˜1279 g/mol, respectively. The PCL4MP has a higher molecular weight than the other components in the resin by a factor of three and consequently lower reactivity. Therefore, the incorporation is slower, and large compositional percentages result in a decreased crosslink density.

All resin formulations display onset glass transition temperatures below 37° C. apart from TPP2, likely due to decreased chain mobility from the urethane groups and low conversion. TPP3 contains only small monomers and displays the characteristics of a uniform network, evidenced by the sharp transition in the storage modulus (E′) curve. A sharp transition indicates that the network contains fewer network imperfections often caused by dangling chains or loops that absorb energy and are characteristic of a broad, shallow transition in DMTA⁵⁶. Additionally, it has been shown that thiol methacrylate networks of similar composition increase conversion over pure methacrylate systems. The increase is attributed to chain transfer associated with the thiyl radical that delays vitrification and mitigates the impact of oxygen inhibition.

The tan δ curves of TPP1a/b/c, TPP2, and TPP4 displayed peaks with relatively low intensity and large breadth, as best seen in FIG. 3. These characteristics are related to the morphology of the polymer network and describe heterogeneous networks with limited chain mobility. Heterogeneity is commonly seen in methacrylate-based networks as vitrification causes microgels and reactive radicals to become trapped. Notably, the tan δ curve for TPP3 is sharp with high intensity. The sharp peak corresponds to a more homogenous network substantiated by the storage modulus curve. The distinct transitions may be due to having thiols in the network that react and act as chain transfer agents, leading to higher conversions. Although TPP1a and TPP4 have similar compositions to TPP3, the long PCL chains within TPP1a and TPP4 create a more heterogeneous network through increased chain entanglements and lower reactivity. Furthermore, the intensity of the tan δ peak for TPP3 indicates that the network favors viscous effects and can dissipate more energy over the other formulations. The sudden change in chain mobility is a desirable property for space-filling and shape memory applications because it allows the network to change geometry after the T_{g} is exceeded.

Cytotoxicity: Cytotoxicity was performed in accordance with ISO 10993-5. The extractions were performed in triplicate and did not contain cytotoxic moieties. Although several compositions showed viability above 100%, there were no statistical differences from the negative control. ISO 10993-5 establishes cytotoxicity as having cell viability below 70% (also known as the IC30 value). In the case of the thiol-ene based resins, all compositions exceeded this threshold and can be considered non-cytotoxic. FIG. 4 is a graphical representation of cell viability of resin compositions per ISO-10993-5 compared to the acceptance criteria of 70% indicated by the horizontal line. However, it is well understood that unreacted methacrylates are cytotoxic and can lead to undesirable results. Therefore, even under stringent cleaning and post-processing, future characterization will focus on the comprehensive biocompatibility of the material outlined in ISO 10993-1.

Atomic Charge Modeling: The compounds modeled mimic an ideal reaction between one PTMP and four BDMA monomers. The model compounds at different sulfur oxidation states demonstrate a shift in the atomic charge and, therefore, electronegativity. In the non-oxidative state, the electronegativity of the core ester group is higher than the outer ester group. The difference in the baseline values may be attributed to the outer ester group resulting from the reacted methacrylate and the core group as a traditional ester group. It has been established that increased hydrophobicity of methacrylate groups leads to slower hydrolysis rates than ester groups from reacted acrylates. As the oxidation state of the sulfide linkages increases to sulfoxides and sulfones, there is a direct relationship with the atomic charge, FIG. 6. FIG. 6 graphically depicts the theoretical atomic charge (e) of the central carbonyl atoms from the model compound of FIG. 1. The protected, core esters and unprotected outer esters demonstrate an increase in atomic charge as a function of sulfur-oxidation. Additionally, as sulfides undergo oxidation, there is an increase in hydrophilicity due to a shift in electronegativity of the sulfur group to a more negative value.

Conversely, the oxidation leads to a more positive value of the central ester carbonyl for both groups. The change in electronegativity increases the susceptibility of hydrolysis by increasing electrophilicity of the central carbon and a nucleophilic attack. While both groups shift to higher positive values after oxidation, the impact from sulfides to sulphones is greater on the outer group esters changing 5.7% overall compared to the core group changing 1.3%. The proximity of thiols was modulated from the ester group, the relative change in electronegativity was 1.9% and resulted in hydrolysis rates changing from days to weeks.

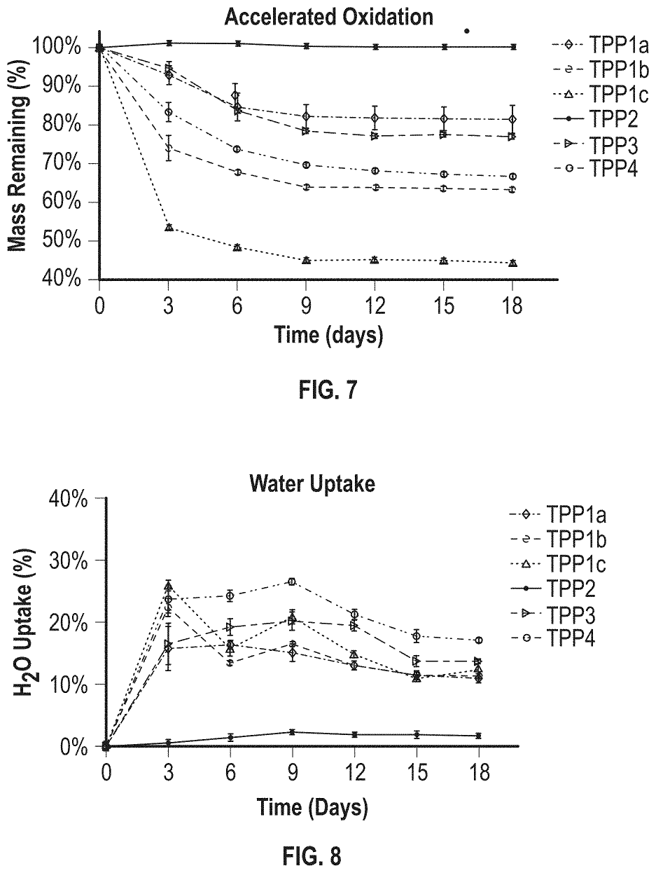

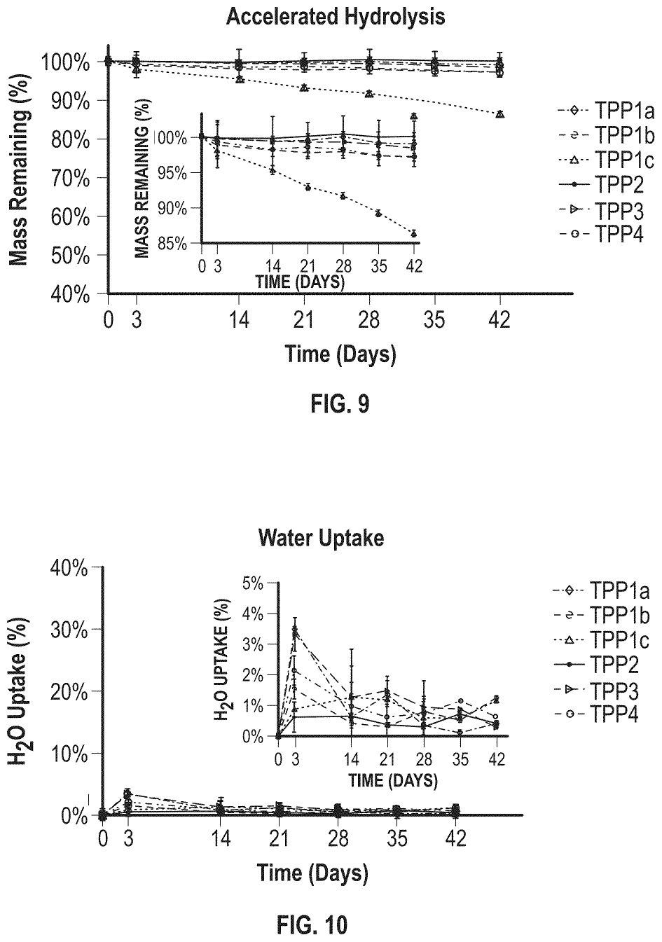

Oxidative Degradation: In vitro accelerated degradation was performed in an oxidative environment to characterize the oxidative response of the polymer. Accelerated oxidation was characterized by rapid changes in mass followed by a plateau for scaffolds that contained sulfide groups, FIGS. 7-10. FIG. 7 illustrates the percent mass loss and FIG. 8 illustrates the water uptake under accelerated oxidative conditions. FIG. 9 illustrates the percent mass loss and FIG. 10 illustrates the water uptake under accelerated hydrolytic conditions. The impact of crosslink density can be seen with TPP1a, TPP1b, and TPP1c as the amount of PCL4MP increases.

Crosslink density is inversely proportional to the molecular weight between crosslinks. As the crosslink density decreases, a higher mass loss is observed, with TPP1c being the highest among all groups and only 44% mass remaining after 18 days in 20% H₂O₂. Conversely, the oxidative environment did not impact the control group, with 100% mass remaining after 18 days in accelerated conditions. The degradation rate was directly related to the ratio of methacrylate to thiol. TPP1a, TPP1b and TPP1c demonstrate this relationship by increased mass loss of 18%, 37% and 56% for thiol content of 30%, 40%, and 50%, respectively.

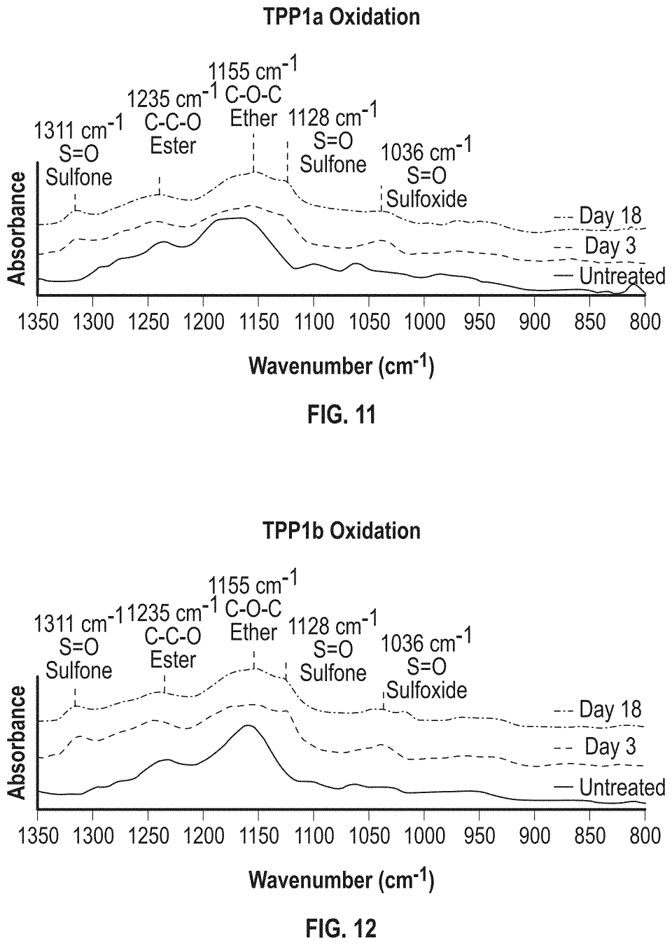

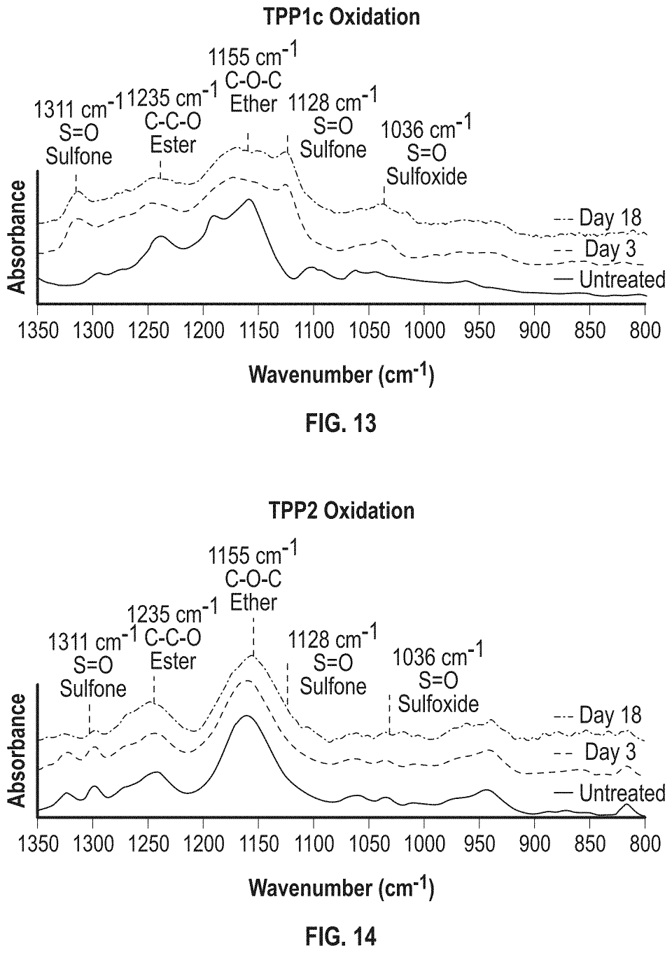

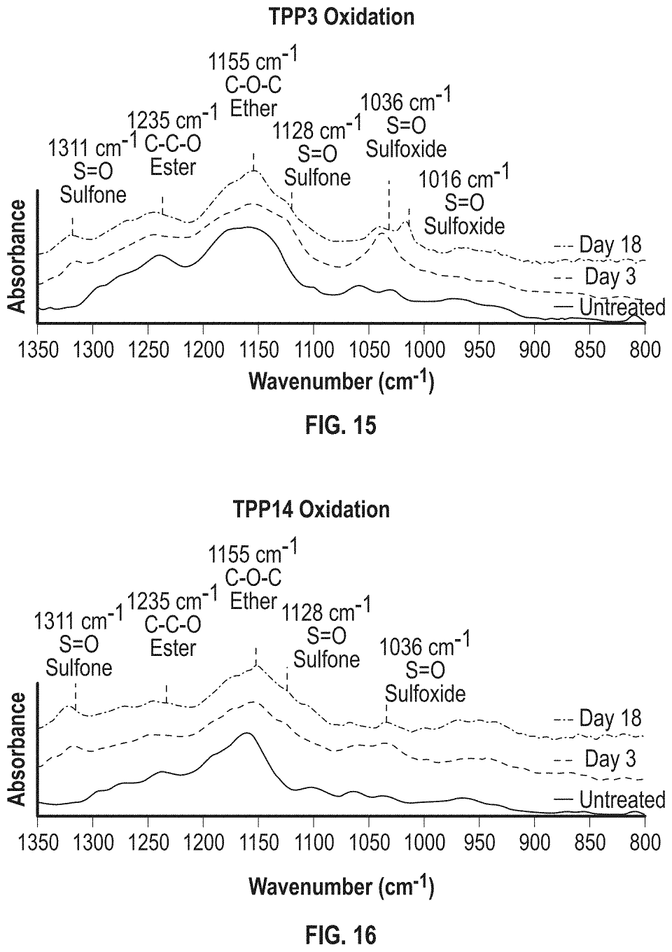

All compositions that contain thiols display various amounts of degradation in the accelerated oxidative media. This phenomenon is attributed to the oxidation of sulfide functional groups. Prior art has demonstrated the conversion of sulfide groups in the presence of H₂O₂. ATR-FTIR confirms the presence of sulfoxide and sulfone groups consistent with the oxidation of sulfides, FIGS. 11-16. FIGS. 11-16 each illustrate a graph of different ATR-FTIR waveforms of thiol-ene photopolymers subjected to accelerated oxidative degradation at 3 days and 18 days compared to untreated control.

In formulations that contain thiol groups, there is evidence of functional oxidation through peaks ca 1036 cm⁻¹, characteristic of sulfoxides and 1311 cm⁻¹ sulfones. The increased hydrolysis is further supported because the water uptake after oxidation initially increases, followed by a plateau and subsequent decrease. The plateau follows the inverse trend of mass loss, indicating it can be attributed to cleavage ester moieties next to sulfur atoms. The degradation of the network increases the molecular weight between crosslinks and allows more water into the polymer matrix. However, because oxidation primarily occurs at the surface, the amount of water uptake is limited. Additionally, once the oxidized polymer chains have been hydrolyzed, the water uptake is expected to decrease. The oxidation and hydrolysis cycles are expected to continue until the polymer matrix is completely degraded. As expected, the control resin, TPP2, containing no sulfide groups, did not undergo any mass loss or surface chemistry changes. The results of the accelerated oxidative degradation confirm that the thiol-ene networks fabricated are oxidatively responsive.

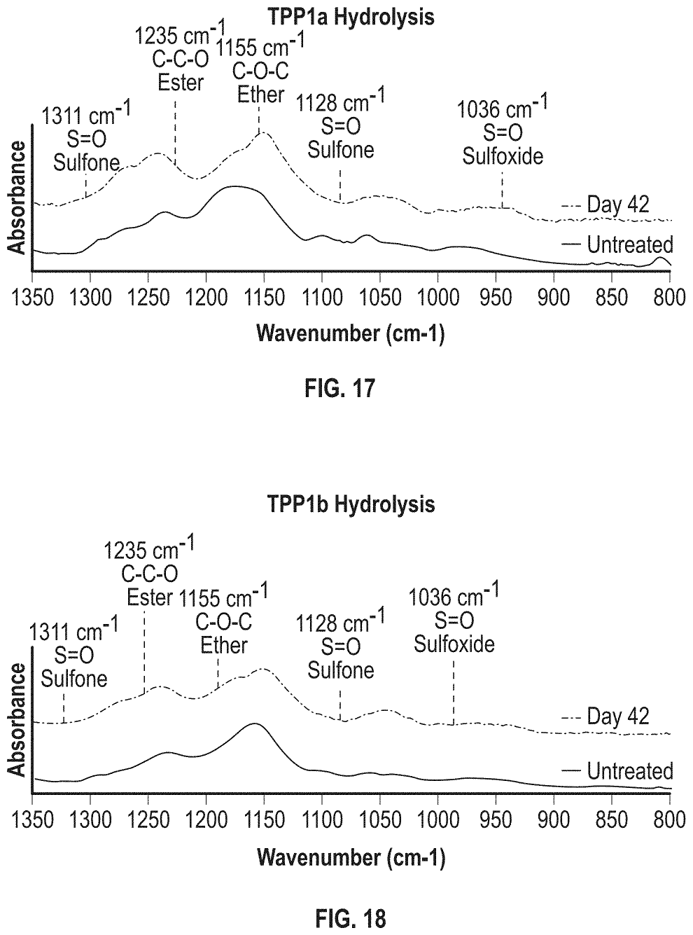

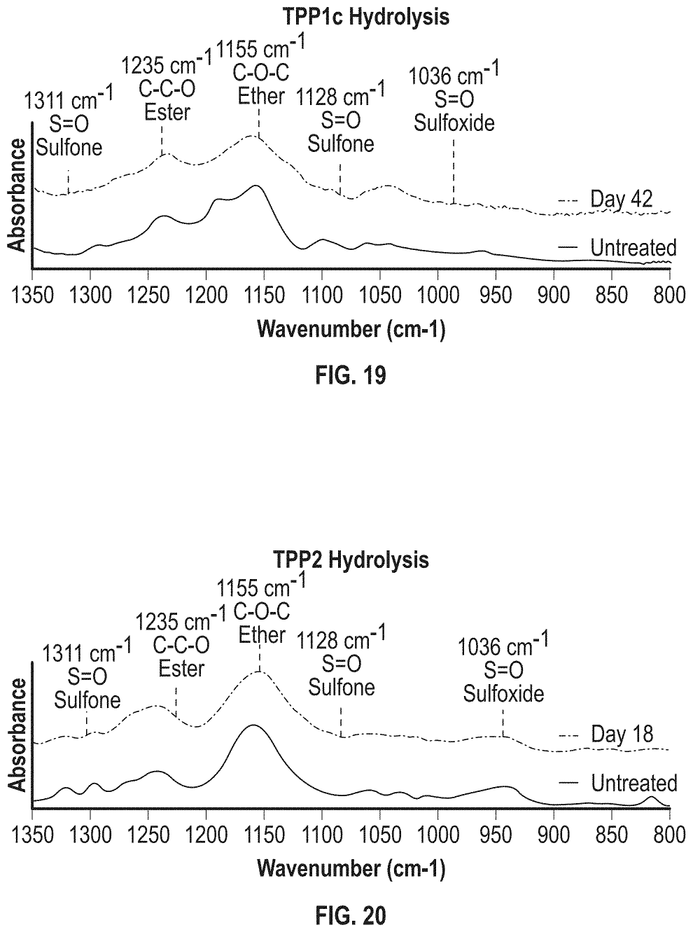

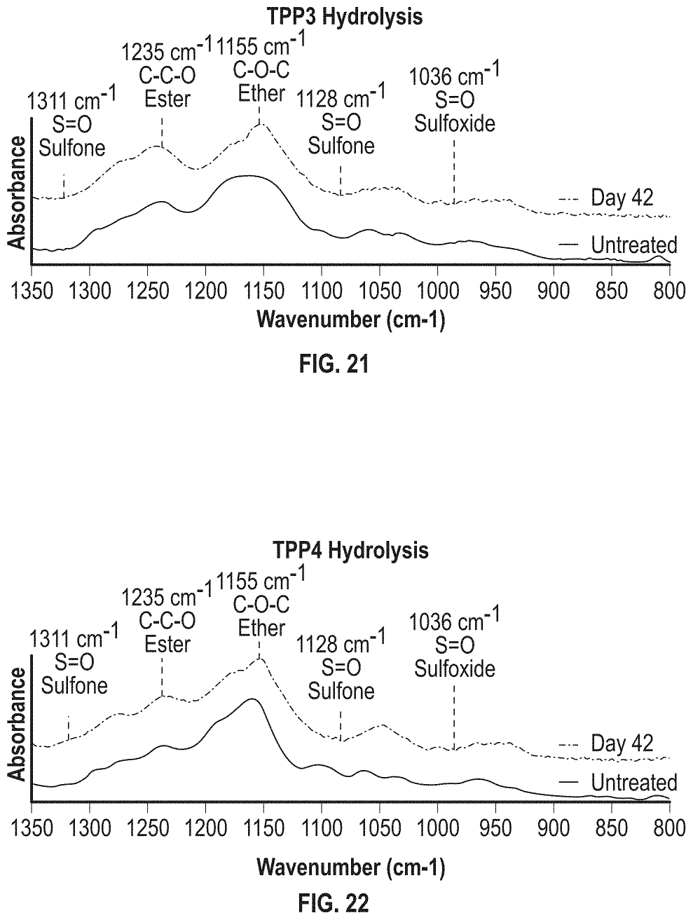

Hydrolytic Degradation: Resin formulations remained essentially unchanged in accelerated hydrolysis in 0.1 M NaOH, FIG. 9. TPP1c displayed the highest amount of degradation after 42 days with 13.6% mass loss. All other formulations were hydrolytically stable over the degradation time. The increased degradation of the TTP1c formulation was attributed to the increase of α-hydroxy esters moieties from the addition of PCL chains. Minor changes in the water uptake further support the absence of structural changes such as the crosslink density or changes to hydrophilicity. Further characterization using ATR-FTIR confirms that no changes have occurred to the chemical structure aside from TPP1c, where ester linkages were cleaved as illustrated in FIGS. 17-22. FIGS. 17-22 show ATR-FTIR waveforms of thiol-ene photopolymers (a-f) subjected to accelerated hydrolytic degradation at 42 days compared to untreated control. The preliminary data for the accelerated hydrolysis also corroborates that degradation of the resins is initiated through oxidation that changes the hydrolytic susceptibility of esters present in the network.

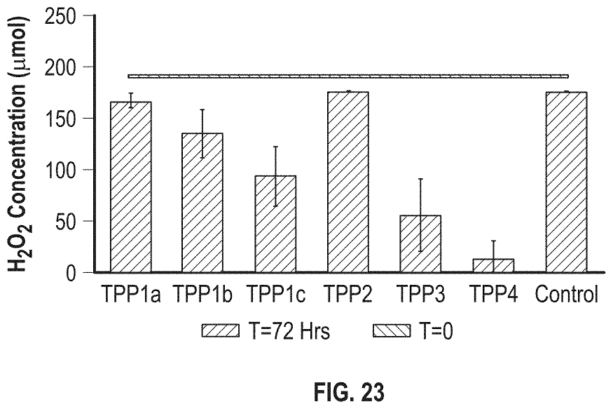

Hydrogen Peroxide Consumption: Increasing thiol content from 70:30 (methacrylate: thiol) to 60:40 and 50:50 correlated to a decrease in hydrogen peroxide concentration as represented by TPP1a, TPP1b, and TPP1c, FIG. 23. FIG. 23 is a graph illustrating the antioxidant activity of thiol-ene photopolymer when submerged in hydrogen peroxide at pathophysiologic levels of 200 μmol. Additionally, H₂O₂ consumption increases in resin compositions that contain shorter chain thiols, such as TPP3 and TPP4. When compared to the control group, the H₂O₂ concentration did not decrease with the thiol-free resin, TPP2. The control blank demonstrated slight degradation with the initial H₂O₂, which is expected as H₂O₂ naturally degrades over time. The consumption of H₂O₂ follows a similar trend seen with oxidative degradation, with more molecules consumed as the amount of thiol content increases. Further studies are needed to assess the scavenging ability of the polymer networks throughout the device's expected lifetime. However, the preliminary assessment of the thiol-ene resin confirms that consumption of H₂O₂ can occur at physiologic levels with reasonable timeframes.

Several thiol-ene based polymer networks were successfully fabricated and characterized. Dynamic mechanical analysis showed that a range of properties was obtainable and overlap with modulus values associated with cancellous bone (>50 MPa). The incorporation of short-chain led to a more homogenous network as evidenced by a shift to a sharp drop in storage modulus and narrow tan δ peak. Additionally, thiol-containing polymer networks demonstrated oxidative dependence via hydrogen peroxide. Furthermore, oxidation of the sulfide linkages increases hydrophilicity and accelerates hydrolysis.

It is hypothesized that this mechanism is attributed to the cleavage of the ester groups in proximity to sulfur atoms. Hydrolysis triggered through oxidative degradation may be beneficial in developing a bone graft as the mechanism is traditionally via surface degradation and may mitigate auto-catalytic amounts of acidic byproducts. Another potential benefit of this mechanism is that the antioxidant capability is staggered through differing reaction kinetics between sulfide to sulfoxide and sulfoxide to sulfone. Coupled with diffusion-limited reactions with ROS, the effectiveness of this polymer system may achieve a sustained antioxidant effect tailored to the relative concentration of hydrogen peroxide. When subjected to physiologically relevant levels, the investigated thiol-ene polymers were shown to readily sequester hydrogen peroxide leading to a decrease in overall ROS levels.

These stimuli-responsive polymers demonstrate effectiveness as biomaterials where high levels of ROS are present and a localized approach is required, such as MPJ salvage procedures.

An increase in pro-inflammatory factors and the high presence of reactive oxygen species (ROS) present in diabetics are linked to increased osteoclastic differentiation and lower fusion rates. To this end, provided is a clinically relevant bone graft to promote bone fusions in patients with neuropathic comorbidities. The incorporation of thiol-ene networks for bone scaffolds increases alkaline phosphatase and osteocalcin expression over poly(ε-caprolactone) hydroxyapatite scaffolds (PCL: HA). Furthermore, thiol-ene networks can act as antioxidants. Sulfide linkages within the network have an ability to consume radical oxygen to create sulfoxide and sulfone groups. These unique properties of thiol-ene networks make them a promising candidate as bone grafts for diabetic patients and patients with other neuropathic comorbidities.

General Interpretive Principles for the Present Disclosure

Various aspects of the novel systems, apparatuses, and methods are described more fully hereinafter with reference to the accompanying drawings. The teachings disclosure may, however, be embodied in many different forms and should not be construed as limited to any specific structure or function presented throughout this disclosure. Rather, these aspects are provided so that this disclosure will be thorough and complete, and will fully convey the scope of the disclosure to those skilled in the art. Based on the teachings herein one skilled in the art should appreciate that the scope of the disclosure is intended to cover any aspect of the novel systems, apparatuses, and methods disclosed herein, whether implemented independently of or combined with any other aspect of the disclosure. For example, a system or an apparatus may be implemented, or a method may be practiced using any one or more of the aspects set forth herein. In addition, the scope of the disclosure is intended to cover such a system, apparatus or method which is practiced using other structure, functionality, or structure and functionality in addition to or other than the various aspects of the disclosure set forth herein. It should be understood that any aspect disclosed herein may be set forth in one or more elements of a claim. Although some benefits and advantages of the preferred aspects are mentioned, the scope of the disclosure is not intended to be limited to particular benefits, uses, or objectives. The detailed description and drawings are merely illustrative of the disclosure rather than limiting, the scope of the disclosure being defined by the appended claims and equivalents thereof.

With respect to the use of plural vs. singular terms herein, those having skill in the art can translate from the plural to the singular and/or from the singular to the plural as is appropriate to the context and/or application. The various singular/plural permutations may be expressly set forth herein for sake of clarity.

When describing an absolute value of a characteristic or property of a thing or act described herein, the terms “substantial,” “substantially,” “essentially,” “approximately,” and/or other terms or phrases of degree may be used without the specific recitation of a numerical range. When applied to a characteristic or property of a thing or act described herein, these terms refer to a range of the characteristic or property that is consistent with providing a desired function associated with that characteristic or property.

In those cases where a single numerical value is given for a characteristic or property, it is intended to be interpreted as at least covering deviations of that value within one significant digit of the numerical value given.

If a numerical value or range of numerical values is provided to define a characteristic or property of a thing or act described herein, whether or not the value or range is qualified with a term of degree, a specific method of measuring the characteristic or property may be defined herein as well. In the event no specific method of measuring the characteristic or property is defined herein, and there are different generally accepted methods of measurement for the characteristic or property, then the measurement method should be interpreted as the method of measurement that would most likely be adopted by one of ordinary skill in the art given the description and context of the characteristic or property. In the further event there is more than one method of measurement that is equally likely to be adopted by one of ordinary skill in the art to measure the characteristic or property, the value or range of values should be interpreted as being met regardless of which method of measurement is chosen.

It will be understood by those within the art that terms used herein, and especially in the appended claims (e.g., bodies of the appended claims) are intended as “open” terms unless specifically indicated otherwise (e.g., the term “including” should be interpreted as “including but not limited to,” the term “having” should be interpreted as “having at least,” the term “includes” should be interpreted as “includes but is not limited to,” etc.).

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

In those instances where a convention analogous to “at least one of A, B, and C” is used, such a construction would include systems that have A alone, B alone, C alone, A and B together without C, A and C together without B, B and C together without A, as well as A, B, and C together. It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include A without B, B without A, as well as A and B together.”

Various modifications to the implementations described in this disclosure can be readily apparent to those skilled in the art, and generic principles defined herein can be applied to other implementations without departing from the spirit or scope of this disclosure. Thus, the disclosure is not intended to be limited to the implementations shown herein but is to be accorded the widest scope consistent with the claims, the principles and the novel features disclosed herein. The word “exemplary” is used exclusively herein to mean “serving as an example, instance, or illustration.” Any implementation described herein as “exemplary” is not necessarily to be construed as preferred or advantageous over other implementations.

Certain features that are described in this specification in the context of separate implementations also can be implemented in combination in a single implementation. Conversely, various features that are described in the context of a single implementation also can be implemented in multiple implementations separately or in any suitable sub-combination. Moreover, although features can be described above as acting in certain combinations and even initially claimed as such, one or more features from a claimed combination can in some cases be excised from the combination, and the claimed combination can be directed to a sub-combination or variation of a sub-combination.

The methods disclosed herein comprise one or more steps or actions for achieving the described method. The method steps and/or actions may be interchanged with one another without departing from the scope of the claims. In other words, unless a specific order of steps or actions is specified, the order and/or use of specific steps and/or actions may be modified without departing from the scope of the claims.

What is claimed is:

1. A method of performing arthrodesis in an individual in need thereof, comprising:
providing an artificial bone graft, said bone graft comprising an effective amount of a methacrylate monomer and an effective amount of a thiol-containing macromer; and
implanting said artificial bone graft;
wherein said bone graft possesses antioxidant properties; and
wherein the bone graft scavenges reactive oxygen species and reduces osteoclastic activity at the fusion site.

2. The method of claim 1, wherein said individual is a Type II diabetic.

3. The method of claim 1, wherein said individual is osteoporotic.

4. The method of claim 1, wherein said arthrodesis is an MPJ fusion procedure.

5. The method of claim 1, wherein said methacrylate monomer is 1, 4-budanediol-dimethacrylate, diurethane dimethacrylate, or combinations thereof.

6. The method of claim 1, wherein said thiol-containing macromer is a multi-functional mercaptopropionate, mercaptoacetate, or combinations thereof.

7. The method of claim 1, wherein the methacrylate monomer and thiol-containing macromer are present at a ratio of 50:50.

8. The method of claim 1, wherein the methacrylate monomer and thiol-containing macromer are present at a ratio of 60:40.

9. The method of claim 1, wherein the methacrylate monomer and thiol-containing macromer are present at a ratio of 70:30.

10. An oxidatively responsive polymeric scaffold for promoting bone fusion, comprising:
an effective amount of a methacrylate monomer; and
an effective amount of a thiol-containing macromer;
wherein said polymeric scaffold comprises sulfide linkages configured to sequester reactive oxygen species molecules; and
wherein said scaffold has a degradation rate which is complementary to the rate of bone healing.

11. The scaffold of claim 10, wherein said methacrylate monomer is 1,4-butanediol dimethacrylate.

12. The scaffold of claim 10, wherein said methacrylate monomer is diurethane dimethacrylate.

13. The scaffold of claim 10, wherein said thiol-containing macromer is a multi-functional mercaptopropionate, mercaptoacetate, or combinations thereof.

14. The scaffold of claim 10, further comprising a bioactive agent coupled to a thiolated group.

15. The scaffold of claim 14, wherein said bioactive agent is an Arginine-Glycine-Aspartate peptide sequence.

16. The scaffold of claim 14, wherein said bioactive agent is a TGF-β.

17. The scaffold of claim 10, wherein the methacrylate monomer and thiol-containing macromer are present at a ratio of 50:50.

18. The scaffold of claim 10, wherein the methacrylate monomer and thiol-containing macromer are present at a ratio of 70:30.

19. The scaffold of claim 10, wherein the methacrylate monomer and the thiol-containing macromer are present at a ratio of 60:40.

20. The scaffold of claim 10, wherein said scaffold is formulated as a bone graft.

21. The scaffold of claim 10, wherein said scaffold is formulated as an injectable gel.

22. The scaffold of claim 10, wherein said scaffold is formulated as an artificial joint surface comprising a hydrophilic layer within a matrix of the polymer.

23. The scaffold of claim 10, further comprising a plurality of pores having an average pore size between about 80 μm and 300 μm.
